(19) United States
(12) Patent Application Publication
(10) Pub. No.: US 2015/0112169 A1
(43) Pub. Date: Apr. 23, 2015

(54) FINGER-PLACEMENT SENSOR

(71) Applicants: Marcelo M. Lamego, Cupertino, CA (US); Hung The Vo, Fountain Valley, CA (US); Cristiano Dalvi, Lake Forest, CA (US); Sean Merritt, Lake Forest, CA (US)

(72) Inventors: Marcelo M. Lamego, Cupertino, CA (US); Hung The Vo, Fountain Valley, CA (US); Cristiano Dalvi, Lake Forest, CA (US); Sean Merritt, Lake Forest, CA (US)

(21) Appl. No.: 14/214,633

(22) Filed: Mar. 14, 2014

Related U.S. Application Data
(60) Provisional application No. 61/785,487, filed on Mar. 14, 2013.

(51) Int. Cl. A61B 5/00; A61B 5/145; A61B 5/1495; A61B 5/1455

(52) U.S. Cl. CPC A61B 5/6838; A61B 5/1455; A61B 5/14532; A61B 5/1495
USPC 600/316

(57) ABSTRACT

A finger-placement sensor fixture aligns and removably secures a finger to a sensor pad of a reusable finger-clip optical sensor so as to assure the finger is repeatably aligned between the sensors emitters and detectors and that the finger stays aligned during a test procedure. The sensor fixture has a sensor pad configured to removably install within a sensor clip. The sensor pad has a sensor cavity custom molded to the shape of an individual's fingertip. A plurality of metal strips are embedded within the sensor pad. A plurality of magnets are embedded within the sensor clip. The sensor pad metal strips are configured to align with the sensor clip magnets so that the sensor pad can be removed, disposed of, replaced and consistently aligned with the sensor clip.

### PRIORITY CLAIM TO RELATED PROVISIONAL APPLICATION

The present application claims priority benefit under 35 U.S.C. §119(e) to U.S. Provisional Patent Application Ser. No. 61/785,487 filed Mar. 14, 2013, titled Finger-Placement Sensor Fixture. The above-cited provisional patent application is hereby incorporated in its entirety by reference herein.

### BACKGROUND OF THE INVENTION

Noninvasive physiological monitoring systems for measuring constituents of circulating blood have advanced from basic pulse oximeters capable of measuring blood oxygen saturation to advanced blood parameter monitors capable of measuring various blood constituents. A basic pulse oximeter typically includes an optical sensor, a monitor for processing sensor signals and displaying results and a cable electrically interconnecting the sensor and the monitor. A basic pulse oximetry sensor typically has a red wavelength light emitting diode (LED), an infrared (IR) wavelength LED and a photodiode detector. The LEDs and detector are attached to a patient tissue site, such as a finger. The cable transmits drive signals from the monitor to the LEDs, and the LEDs respond to the drive signals to transmit light into the tissue site. The detector generates a photoplethysmograph signal responsive to the emitted light after attenuation by pulsatile blood flow within the tissue site. The cable transmits the detector signal to the monitor, which processes the signal to provide a numerical readout of oxygen saturation (SpO₂) and pulse rate, along with an audible pulse indication of the person's pulse. The photoplethysmograph waveform may also be displayed.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, Calif. and are incorporated in their entirety by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entirety by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are capable of measuring various blood parameters in addition to SpO₂, such as total hemoglobin and carboxyhemoglobin to name a few. Advanced blood parameters measurement systems are described in at least U.S. Pat. No. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. No. 7,957,780, filed Mar. 1, 2006, titled Physiological Parameter Confidence Measure and U.S. Pat. No. 8,224,411, filed Mar. 1, 2006, titled Noninvasive Multi-Parameter Patient Monitor, all assigned to Cercacor Laboratories, Inc., Irvine, Calif. (“Cercacor”) and all incorporated in their entirety by reference herein. An advanced parameter measurement system that includes acoustic monitoring is described in U.S. Pat. Pub. No. 2010/0274099, filed Dec. 21, 2009, titled Acoustic Sensor Assembly, assigned to Masimo and incorporated in its entirety by reference herein.

Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-7™ and Rad-57™ monitors, all available from Masimo. Advanced parameter measurement systems may also include acoustic monitoring such as acoustic respiration rate (RRa™) using a Rainbow Acoustic Sensor™ and Rad-87™ monitor, available from Masimo. Such advanced pulse oximeters, low noise sensors and advanced parameter systems have gained rapid acceptance in a wide variety of medical applications, including surgical wards, intensive care and neonatal units, general wards, home care, physical training, and virtually all types of monitoring scenarios.

### SUMMARY OF THE INVENTION

A finger-placement sensor repeatably aligns a finger within a reusable finger-clip optical sensor and in particular between the sensor emitters and detectors so as to obtain consistent blood parameter test results. Consistent finger-sensor alignment is particularly advantageous when making noninvasive blood glucose measurements with an optical sensor. The sensor fixture integrates a custom finger mold for each individual. In an embodiment, the custom mold is repeatably aligned within an optical sensor clip using metal tabs embedded in the mold and corresponding rare earth magnets disposed within the sensor clip.

One aspect of a finger-placement sensor is a fixture that aligns and removably secures a finger to a sensor pad of a reusable finger-clip optical sensor so as to assure the finger is repeatably aligned between the sensors emitters and detectors and that the finger stays aligned during a test procedure. The sensor fixture comprises a custom sensor pad configured to removably install within a sensor clip and the sensor pad has a sensor cavity that conforms to the shape of an individual's fingertip. In various embodiments, the finger-placement sensor fixture has a top sensor pad that conforms to the fingernail-side of a selected finger of the individual. Metal strips are embedded within the top sensor pad so as to aid the alignment of the top sensor pad within an emitter shell of the sensor clip. Magnets are embedded within the emitter shell so as to align the metal strips with respect to the magnets. A bottom sensor pad conforms to the finger pad-side of the selected finger. A second plurality of magnets are embedded with a detector shell of the sensor clip. A second plurality of metal strips are embedded within the bottom sensor pad so as to aid the alignment of the bottom sensor pad within the detector shell of the sensor clip.

Another aspect of a finger-placement sensor is a method for consistently aligning a fingertip within a reusable optical sensor that removably clips onto the fingertip so as to noninvasively measure constituents of blood flow within the fingertip. The method comprises physically analyzing potential measurement sites as suitable for optical sensor measurements, manufacturing a sensor fixture and evaluating the sensor fixture. In various embodiments, physically analyzing comprises eliminating finger sites that have congenital defects, prior injuries or unusual shapes and sizes. Manufacturing a sensor fixture comprises generating at least one of a hand mold or an optical scan finger image. Evaluating the sensor fixture comprises comparing a series of optical sensor measurements utilizing the sensor fixture with test strip measurements taken over a predetermined period of time and determining if the variance of the optical sensor measurements compared with the test strip measurements are within predetermined limits. Manufacturing a sensor fixture further comprises creating an injection mold based upon the at least one of a hand mold or an optical scan finger image. Manufacturing a sensor fixture further comprises molding a sensor pad from the injection mold and embedding at least one metal alignment strip within the sensor pad.

A further aspect of a finger-placement sensor fixture attachable within at least one shell portion of a reusable optical sensor is a sensor pad means for clamping a fingertip within an optical sensor, a finger mold means for conforming the sensor pad to the shape of the fingertip and a magnetic means for aligning the sensor pad within the optical sensor. In various embodiments, the sensor pad means comprises a top sensor pad means for stabilizing the fingernail side of a fingertip within an optical sensor. The finger mold means comprises at least one of an injection mold means or an optical scan means for capturing a specific size and shape of a particular patient's fingertip. The magnetic means comprises a rare earth magnetic means for creating an first alignment object within a sensor clip shell and a metal strip means for creating a second alignment object within the sensor pad. A bottom sensor pad means stabilizes the fingertip side of a fingertip within the optical sensor.

### BRIEF DESCRIPTION OF THE DRAWINGS

FIG. 1 is a perspective illustration of a finger-placement sensor fixture attachable to a sensor clip so as to establish and maintain repeatable finger placement within a reusable optical sensor;

FIG. 2 is a flow diagram of a customized reusable sensor process for a diabetic patient;

FIGS. 3A-B are wire-frame illustrations of an optical finger scan for creating a finger-placement sensor fixture;

FIGS. 4A-D are top, side, front and perspective views, respectively, of an injection mold for creating a finger-placement sensor fixture;

FIGS. 5A-C are perspective views of three individualized finger-placement sensor fixtures; and

FIG. 6 an exploded side view of a optical sensor embodiment having a finger-placement sensor fixture.

### DETAILED DESCRIPTION OF THE PREFERRED EMBODIMENTS

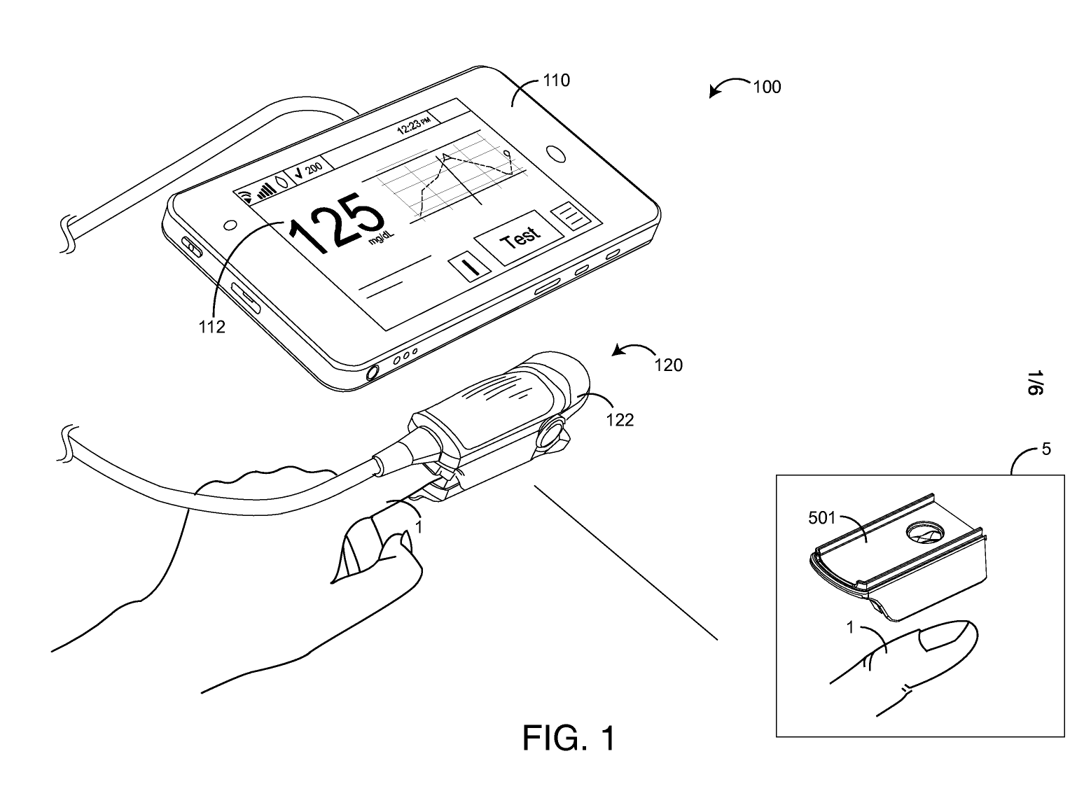

FIG. 1 generally illustrates a physiological monitoring system 100 that utilizes a finger-placement sensor fixture 501. The monitoring system 100 includes a blood parameter monitor 110 and an optical sensor 120 configured to noninvasively measure and display a patient's blood glucose level among other parameters. In an embodiment, the sensor 120 attaches to a person's finger 1 so as to illuminate the finger with optical radiation, which is detected after attenuation by fingertip blood flow. The sensor communicates these optical measurements of blood attenuation, along with other sensor data such as sensor position and temperature, to the monitor 110. The monitor 110 calculates and displays blood parameter measurements 112 accordingly. A physiological monitoring system is described in U.S. patent application Ser. No. 13/308,461 titled Handheld Processing Device Including Medical Applications For Minimally And Non Invasive Glucose Measurements, filed Nov. 30, 2011, assigned to Cercacor Laboratories Inc. (“Cercacor”), and incorporated in its entirety by reference herein. A reusable optical sensor is described in U.S. patent application Ser. No. 13/473,477 titled Personal Health Device, filed May 16, 2012, assigned to Cercacor and incorporated in its entirety by reference herein.

As shown in FIG. 1, and in particular inset 5 therein, optical sensor measurements as described above are sensitive to finger placement between top and bottom sensor pads. A finger-placement sensor fixture 501 is attachable within a sensor clip 122 so as to establish and maintain repeatable finger placement within a reusable optical sensor 120. In an embodiment, the finger-placement sensor fixture 501 replaces one or both of generic top and bottom sensor pads with a customized pad specifically molded to an individual's finger so as to advantageously improve finger placement repeatability.

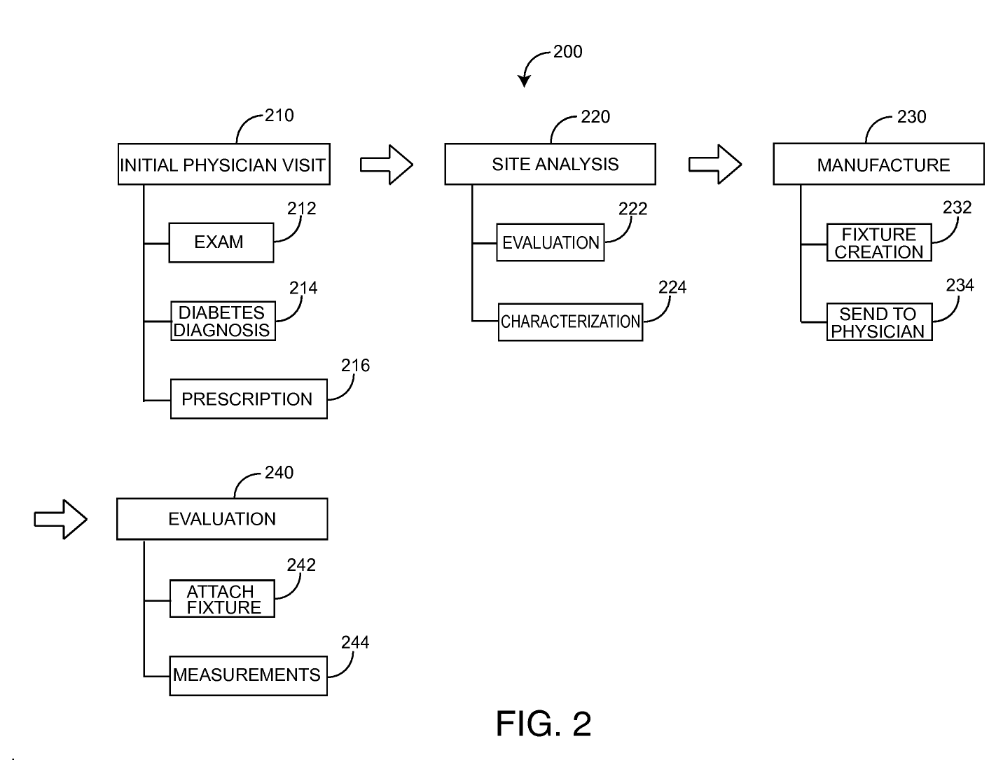

FIG. 2 illustrates a customized reusable sensor process 200 for a diabetic patient or other user. A patient initially visits a physician office 210, such as an endocrinologist or an internal medicine specialist. The physician conducts an exam 212 and various tests. Based upon the exam 212 and test results, the physician makes a diagnosis 214 that the patient has type 1 or type 2 diabetes or other conditions requiring regular or frequent blood constituent monitoring. As a result, the physician informs the patient that they need to frequently monitor their blood glucose levels or other blood parameters as part of a regime for controlling those levels. Accordingly, the physician prescribes noninvasive monitoring technology 216 as an alternative to frequent blood sampling with lances and test strips.

As shown in FIG. 2, following the initial visit 210, the physician or a trained member of the physician's staff conducts a site analysis 220. First, during a site evaluation 222, the physician/staff member identifies areas of the patient's fingers or hands that are potential measurement sites 222. This may involve close physical examination of those patient areas and optical sensor measurements to name a few. For example, some finger sites may be unsuitable for optical sensor measurements due to congenital defects, prior injuries, unusual sizes and shapes, etc. Next, the chosen measurement site is characterized 224 by using photographic techniques, optical scanning (visible/IR) or by taking a physical mold of the measurement site or sites.

Also shown in FIG. 2, the physician site analysis 220 results are then transmitted to a manufacturer 230 so as to create sensor fixtures 232. The sensor fixtures are then shipped back to the requesting physician 234. In particular, the manufacturer 230 uses the physician's scans or molds to create a supply of low-cost, patient-customized, disposable sensor fixtures 232 that each perfectly fit a particular measurement site of a specific patient.

Further shown in FIG. 2, the physician provides each patient with a physiological measurement system 100 and a supply of customized disposable sensor fixtures 232 to install as needed within an optical sensor 120. These sensor fixtures 501 (FIG. 1), when installed within an optical sensor, advantageously allow highly repeatable sensor placement on each specific patient, allowing very accurate and repeatable noninvasive measurements, such as blood glucose, to be taken in lieu of frequent and painful lancing and blood draws necessary for test strip measurements. In an embodiment, these patient-customized, disposable sensor fixtures 501 (FIG. 1) are used in combination with an optically neutral gel placed on the patient measurement site to further enhance the repeatability and accuracy of noninvasive optical sensor measurements of blood glucose.

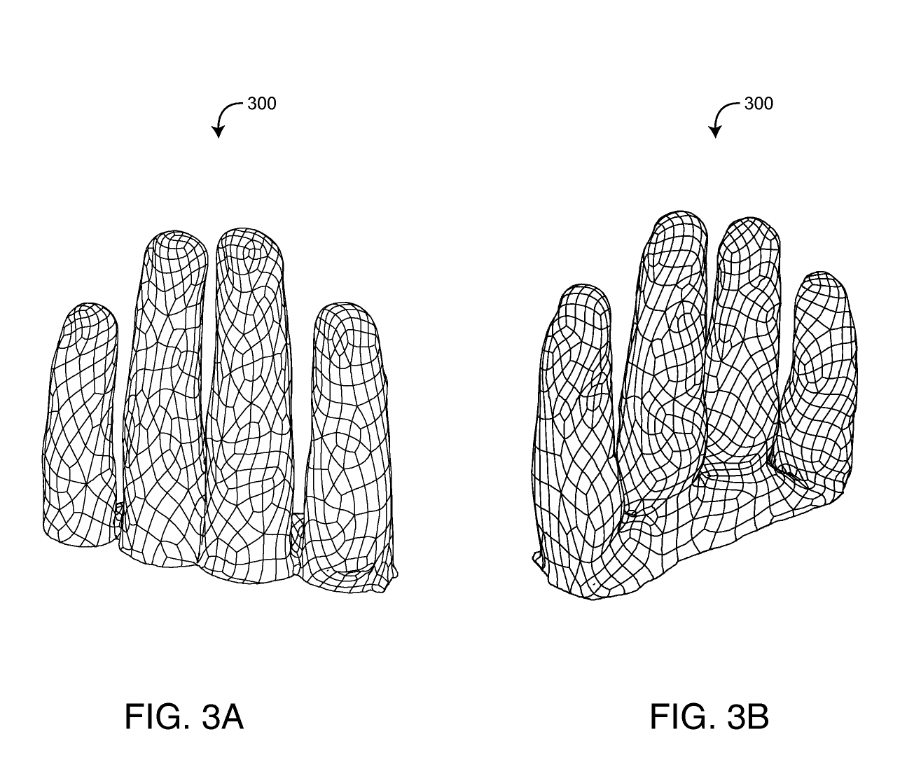

FIGS. 3-5 illustrate various specific aspects and embodiments of site analysis 220 (FIG. 2) and manufacture 230 (FIG. 2), as described with respect to FIG. 2, above. FIGS. 3A-B illustrate wire-frame finger images 300 generated with an optical scan (video camera, stereo camera or snapshot camera imaging) or a physical mold of a patient's hand performed at a physician's office during an initial physician visit.

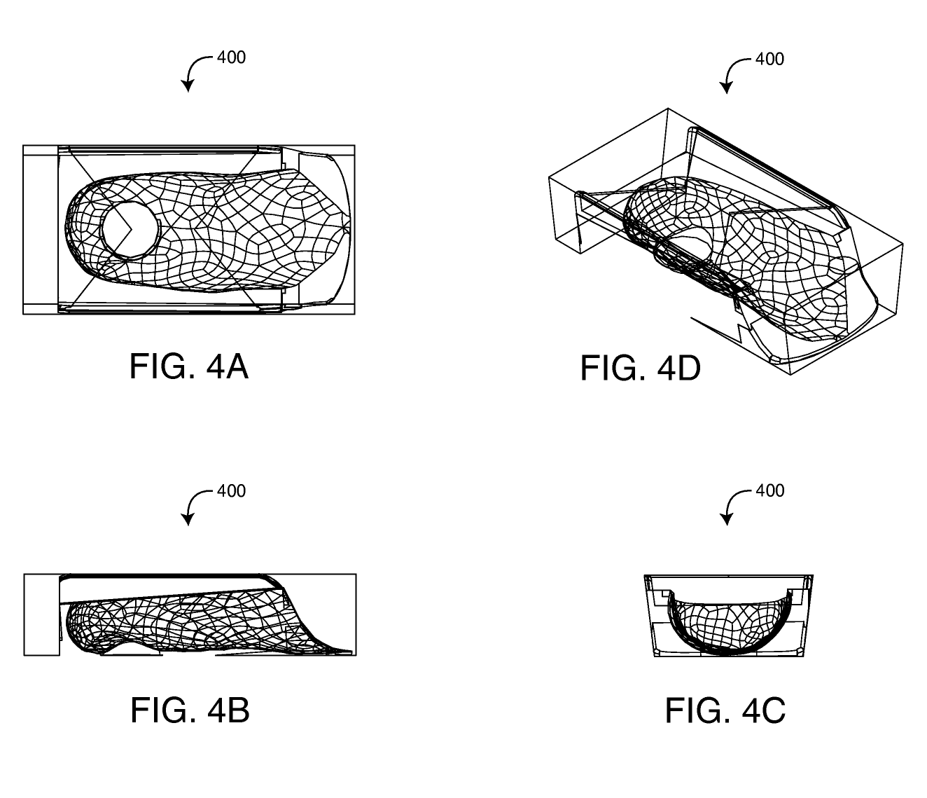

FIGS. 4A-D illustrate an injection mold 400 generated from an optical scan or hand mold for creating a finger-placement sensor fixture. The injection mold 400 is a negative of a selected finger generated from the optical scan finger images 300 (FIGS. A-B). A particular one of a patient's fingers may be selected for the injection mold process based upon the physician's site analysis 220 (FIG. 2). In this example, the injection mold is for a top sensor pad 501 (FIG. 1) that fits the fingernail side of a patient's finger. In other embodiments, an injection mold is made for a bottom sensor pad fitting the finger-pad side of a patient's finger, or for both top and bottom pads.

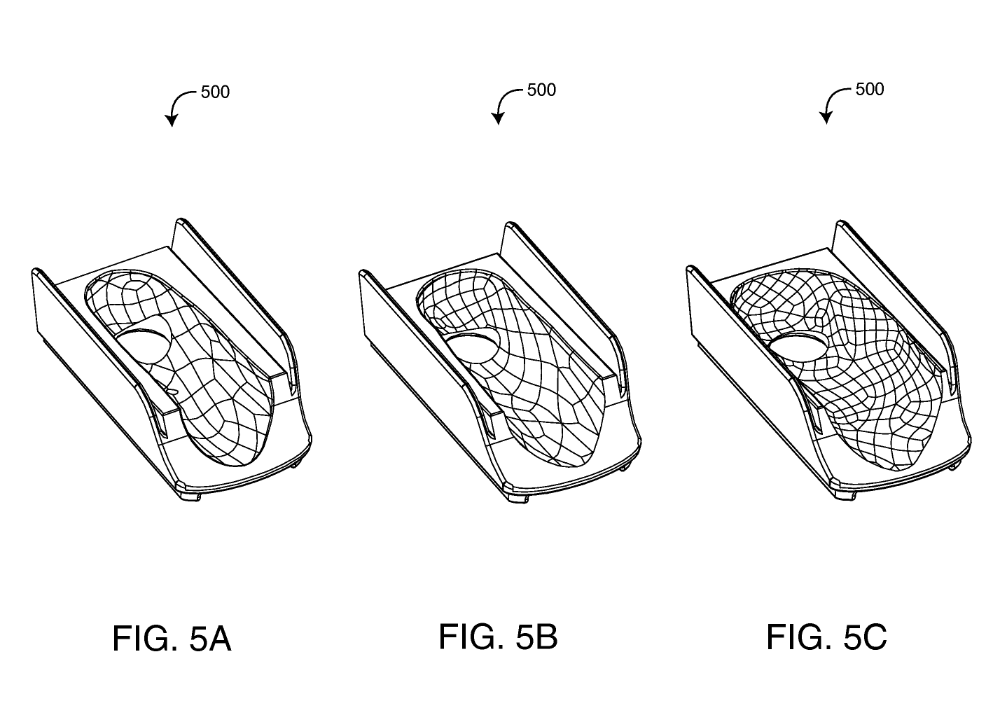

FIGS. 5A-C illustrate finger-placement sensor fixtures 500 for three different individuals, where each sensor fixture 500 is configured as a top sensor pad of an optical sensor 120 (FIG. 1). Advantageously, each sensor fixture 500 is configured to closely conform to the size and shape of a particular individual's finger so that each time that individual takes a sensor measurement, their finger is repeatably positioned within the sensor relative to the optical sensor emitters and detectors each time glucose or other physiological parameter is noninvasively measured. In an embodiment, small metal strips are embedded in the sensor fixture 500 corresponding to small rare earth magnets embedded in the sensor clip so that the disposable sensor fixtures can be replaced in a repeatable and consistent position within the sensor clip 122 (FIG. 1).

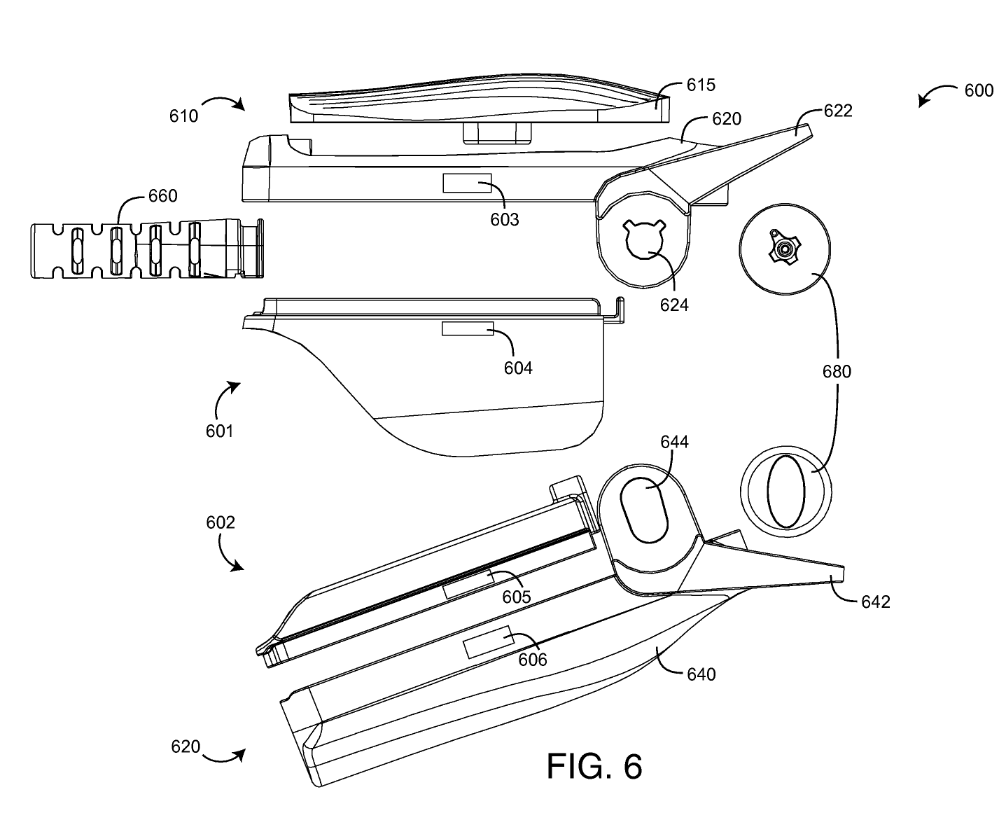

FIG. 6 illustrates a sensor 600 having one or more sensor pads 601, 602 advantageously configured as customized finger-placement sensor fixtures. The sensor 600 has an emitter section 610 that is pivotably connected with a detector section 620 around hinge pins 680, which capture a hinge spring (not shown) that urges the sensor 600 to a closed position. Together, a top grip 622 and a bottom grip 642 form clip grips that press-to-open and release-to-close. The emitter section 610 has a heat sink 615, an emitter shell 620 and a top sensor pad 601. The detection section 620 has a detector shell 640 and a bottom sensor pad 602. A bend relief 660 is captured between the emitter shell 620 and top sensor pad 601 and receives a sensor cable (not shown).

As shown in FIG. 6, advantageously, the top sensor pad 601 removably attaches to the emitter shell 620, and the bottom sensor pad 602 removably attaches to the detector shell 640. In this manner, either the top sensor pad 601, the bottom sensor pad 602 or both may be customized as finger-placement sensor fixtures, as described above.

Further shown in FIG. 6, a pair of top magnets 603 are imbedded on both sides of and within the emitter shell 620. A pair of bottom magnets 606 are imbedded on both sides of and within the detector shell 640. A pair of top metal strips 604 are imbedded on both sides of and within the top sensor pad 601 so as to generally align with the top magnets 603. A pair of bottom metal strips 606 are imbedded on both sides of and within the bottom sensor pad 602 so as to generally align with the bottom magnets 606. Advantageously, the shell magnets 603, 605 strongly attract the sensor pad metal strips 604, 606 so as to consistently align the sensor pads 604, 606 within the sensor 600, allowing these finger-placement sensor fixtures, e.g. 501 (FIG. 1) to maintain consistent finger placement with respect to the sensor emitters and detectors.

A finger-placement sensor fixture has been disclosed in detail in connection with various embodiments. These embodiments are disclosed by way of examples only and are not to limit the scope of the claims that follow. One of ordinary skill in art will appreciate many variations and modifications.

What is claimed is:

1. A finger-placement sensor fixture aligns and removably secures a finger to a sensor pad of a reusable finger-clip optical sensor so as to assure the finger is repeatably aligned between the sensors emitters and detectors and that the finger stays aligned during a test procedure, the sensor fixture comprising:
a custom sensor pad configured to removably install within a sensor clip; and
the sensor pad having a sensor cavity that conforms to the shape of an individual's fingertip.

2. The finger-placement sensor fixture according to claim 1 further comprising a top sensor pad that conforms to the fingernail-side of a selected finger of the individual.

3. The finger-placement sensor fixture according to claim 2 further comprising a plurality of metal strips embedded within the top sensor pad so as to aid the alignment of the top sensor pad within an emitter shell of the sensor clip.

4. The finger-placement sensor fixture according to claim 3 further comprising a plurality of magnets embedded within the emitter shell so as to align the metal strips with respect to the magnets.

5. The finger-placement sensor fixture according to claim 4 further comprising a bottom sensor pad that conforms to the finger pad-side of the selected finger.

6. The finger-placement sensor fixture according to claim 5 further comprising a second plurality of magnets embedded with a detector shell of the sensor clip.

7. The finger-placement sensor fixture according to claim 6 further comprising a second plurality of metal strips embedded within the bottom sensor pad so as to aid the alignment of the bottom sensor pad within the detector shell of the sensor clip.

8. A finger-placement sensor method for consistently aligning a fingertip within a reusable optical sensor that removably clips onto the fingertip so as to noninvasively measure constituents of blood flow within the fingertip comprising:
physically analyzing potential measurement sites as suitable for optical sensor measurements;
manufacturing a sensor fixture; and
evaluating the sensor fixture.

9. The finger-placement sensor method according to claim 8 wherein physically analyzing comprises eliminating finger sites that have congenital defects, prior injuries or unusual shapes and sizes.

10. The finger-placement sensor method according to claim 9 wherein manufacturing a sensor fixture comprises generating at least one of a hand mold or an optical scan finger image.

11. The finger-placement sensor method according to claim 10 wherein evaluating the sensor fixture comprises:
comparing a series of optical sensor measurements utilizing the sensor fixture with test strip measurements taken over a predetermined period of time; and
determining if the variance of the optical sensor measurements compared with the test strip measurements are within predetermined limits.

12. The finger placement method according to claim 11 wherein manufacturing a sensor fixture further comprises creating an injection mold based upon the at least one of a hand mold or an optical scan finger image.

13. The finger placement method according to claim 12 wherein manufacturing a sensor fixture further comprises molding a sensor pad from the injection mold.

14. The finger placement method according to claim 13 wherein manufacturing a sensor fixture further comprises embedding at least one metal alignment strip within the sensor pad.

15. A finger-placement sensor fixture attachable within at least one shell portion of a reusable optical sensor comprising:
a sensor pad means for clamping a fingertip within an optical sensor;
a finger mold means for conforming the sensor pad to the shape of the fingertip; and
a magnetic means for aligning the sensor pad within the optical sensor.

16. The finger-placement sensor fixture according to claim 15 wherein the sensor pad means comprises a top sensor pad means for stabilizing the fingernail side of a fingertip within an optical sensor.

17. The finger-placement sensor fixture according to claim 16 wherein the finger mold means comprises at least one of an injection mold means or an optical scan means for capturing a specific size and shape of a particular patient's fingertip.

18. The finger-placement sensor fixture according to claim 17 wherein the magnetic means comprises:
a rare earth magnetic means for creating an first alignment object within a sensor clip shell; and
a metal strip means for creating a second alignment object within the sensor pad.

19. The finger-placement sensor fixture according to claim 18 further comprising a bottom sensor pad means for stabilizing the fingertip side of a fingertip within the optical sensor.
